(19) United States
(12) Patent Application Publication
(10) Pub. No.: US 2011/0300224 A1
(43) Pub. Date: Dec. 8, 2011

(54) TASTE MASKED DOSAGE FORM OF PHARMACEUTICALLY ACCEPTABLE SALT OF ESCITALOPRAM

(75) Inventors: Deepak Murpani, Pallini (GR); Alexaki Pandora, Pallini (GR)

(73) Assignee: GENEPHARM A.E., Pallini (GR)

(21) Appl. No.: 13/125,736

(22) PCT Filed: Dec. 11, 2009
(86) PCT No.: PCT/EP09/08875

§ 371 Date: Aug. 26, 2011

(30) Foreign Application Priority Data
Oct. 23, 2008 (GR) 20080100696

(51) Int. Cl. A61K 9/14; A61P 43/00; A61K 31/341

(52) U.S. Cl. 424/495; 514/469; 424/490; 424/494; 424/497

(57) ABSTRACT

A taste masked dosage form of pharmaceutical acceptable salt of escitalopram comprising (a) resin complex of pharmaceutical acceptable salt of escitalopram and cationic exchange resin or adsorbing or coating non-pareil seeds or inert particles with a mixture of pharmaceutically acceptable salt of escitalopram, cationic polymer and optionally other polymer(s) or loading non-pareil seeds or inert particles with pharmaceutically salt of escitalopram followed by polymer coating with cationic polymer and optionally other polymer(s); and (b) at least one pharmaceutical excipient.

### TECHNICAL FIELD OF THE INVENTION

The technical field of the invention relates to taste masked dosage form of pharmaceutically acceptable salt of escitalopram and economical processes for the preparation of such taste masked dosage form.

### BACKGROUND OF THE INVENTION

The antidepressant Citalopram was first disclosed in German patent No. 2657013 (hereinafter referred to as '013 patent) assigned to M/s Kefalas. '013 patent also discloses methods for preparing citalopram which is isolated in crystalline form as the hydrobromide, the hydrochloride and the oxalate respectively.

Escitalopram is the S-enantiomer of Citalopram, i.e. (S)-1-[3-(dimethylamino)propyl]-1-(4-fluorophenyl)-1,3-dihydro-5-isobenzofurancarbonitrile. Escitaloram is marketed as Lexapro/Cipralex. Crystalline escitalopram oxalate is disclosed in European patent 347066 (hereinafter referred to as '066 patent) assigned to M/s Lundbeck. Methods for the preparation of escitalopram are also disclosed in the '066 patent. The patent exemplifies tablets containing escitalopram, saccharides, gelatine, povidone, talc and magnesium stearate. This composition will not provide taste masked dosage form of pharmaceutically acceptable salt of escitalopram.

Published application WO 2003/011278 (hereinafter referred to as '278 application) assigned to M/S Lundbeck provides large crystals of escitalopram oxalate having a median particle size of at least 40 microns using a novel crystallization process. '278 application provides film coated tablets of escitalopram oxalate and not the taste masked dosage form of the present invention.

Published United States patent application No. 20070021499 assigned to M/S Lundbeck discloses orodispersible tablets of crystalline base of escitalopram. There is no disclosure on the taste of the exemplified tablets. The free base form is less soluble in water which makes it more palatable than the corresponding salt form with the unacceptable taste. Also, salts of organic compounds are generally more stable than the organic compounds themselves. Salts of escitalopram have melting point greater than 100° C. whereas escitalopram free base melts below 50° C. which creates difficulties during handling and storage. Escitalopram oxalate has a melting point of 147° C. making it user friendly during manufacturing dosage forms. This led us to believe that there exists a need to develop taste masked dosage form of pharmaceutically acceptable salt of escitalopram.

One particular challenge facing the development of orally disintegration dosage form is the unpleasant taste of many drug actives. If not appropriately addressed, this can lead to serious problems of patient compliance.

We have now surprisingly found taste masked dosage form of pharmaceutically acceptable salt of escitalopram and the process of its preparation.

The taste masked dosage form of pharmaceutically acceptable salt of escitalopram according to the present invention ensures excellent stability and bioavailability of escitalopram. The manufacturing process for preparation according to the present invention is simpler and inexpensive.

The taste masked dosage form of pharmaceutically acceptable salt of escitalopram of the present invention is characterized by physiochemical properties suitable for the tablet formulation by wet granulation like possessing good compressibility properties and storage stability.

### OBJECT OF THE INVENTION

The object of the present invention is to provide taste masked dosage form of pharmaceutically acceptable salt of escitalopram and the process of its preparation.

More specifically, the object of the present invention is to provide taste masked dosage form of escitalopram oxalate and the process of its preparation.

### DESCRIPTION OF THE INVENTION

Many very young and elderly patients have trouble in swallowing whole tablets and even capsules. It is therefore desirable to administer drugs to such patients either as a liquid dosage form or as a fast dissolving or orally disintegrating solid dosage form. Fast dissolving or orally disintegrating solid dosage forms, due to their ease of administration and pleasant taste, may encourage patients to adhere to daily medication regimes and therefore provide better compliance. Further, orally disintegrating tablets provide the convenience of a tablet formulation while also allowing the ease of swallowing provided by a liquid formulation. These dosage forms also allow much more accurate dosing than the primary alternative, oral liquid.

Palatability is the most important characteristic to be considered in providing fast dissolving or disintegrating solid dosage forms, or matrix for a drug. This characteristic is important as many drugs have a bitter or otherwise unpalatable taste, which makes such drugs unsuitable for administration as fast dissolving or fast disintegrating dosage forms. In order to mask the taste of pharmaceutically acceptable salt of escitalopram we have developed taste masked resinate, granules and dosage forms for administration of the dosage form to patients.

The present invention is directed to taste masked dosage form of pharmaceutically acceptable salt of escitalopram. The pharmaceutically acceptable salt of escitalopram for the present invention may be selected from hydrochloride, hydrobromide and oxalate, preferably the pharmaceutically acceptable salt is oxalate.

According to one embodiment of the present invention a taste masked dosage form of pharmaceutically acceptable salt of escitalopram is provided. Different techniques are employed for taste masking active ingredients such as addition of effervescent disintegrating agent, polymer coating, resinate complex of active ingredient and the like. The present invention also utilizes some of these techniques.

The taste masked dosage form of pharmaceutically acceptable salt of escitalopram of the present invention may be selected from tablet, soluble tablet, sprinkle granules or powder for reconstitution in a suspension, rapidly disintegrating tablet, orally disintegrating tablet, rapidly disintegrating film, orally disintegrating powder for capsules, suspension or sachets, effervescent tablet, chewable tablet, water dispersible tablet, orodisperisable tablet, chewing gum and suspension.

The term “orally disintegrating”, used in the present invention, means that the pharmaceutical composition disintegrates within 180 seconds as measured by the in vitro disintegrated test according to Ph.Eur.

The present invention provides taste masked orally disintegrating tablet of pharmaceutically acceptable salt of escitalopram.

More particularly, the present invention provides taste masked orally disintegrating tablet of escitalopram oxalate.

According to another embodiment of the present invention, a taste masked dosage form of pharmaceutically acceptable salt of escitalopram may comprise taste masked resinate of pharmaceutically acceptable salt of escitalopram complexed with a cationic ion exchange resin and atleast one pharmaceutical excipient. The resinate may be used as such or as granules in the preparation of dosage form.

The pharmaceutically acceptable salt of escitalopram may be selected form hydrochloride, hydrobromide and oxalate, preferably the pharmaceutically acceptable salt is oxalate.

Ion exchange resins are known as polymeric materials which have the possibility to form weak bonds with drugs having positive charge. They are insoluble polymers which contain acidic or basic functional groups and have the ability to exchange counter ions with aqueous solution surrounding them. In general a drug ion are exchanged. A batch process of complexation is optimized with reference to drug loading (ratio of active and resin), temperature and pH. The typical way of loading active ingredient onto an ion exchanges resin is to dissolve or disperse an acidic or basic, ionizable active ingredient in water, and then mix it with a suitable ion exchange resin.

The complexation between cationic ion exchange resin and the pharmaceutically acceptable salt of escitalopram, hinders the release of pharmaceutically acceptable salt of escitalopram in the mouth, so that the patient does not feel the unpleasant taste of the drug when it is swallowed. When the drug resinate comes into contact with the gastrointestinal fluid, such as the acid of the acid of the stomach, the drug is released from resinate directly into solution and then absorbed in the usual way. The resin passes through the GI tract without being absorbed.

Thus, the resinate, a complex of pharmaceutically acceptable salt of escitalopram with cationic exchange resin, serves as a protective barrier, suppressing release of the active ingredient in the pH environment of the oral cavity.

The cationic ion exchange resin used in the present invention may be selected from
(a) the cationic (strongly acidic) ion exchange resin Amberlite Resin Grade IRP-69 (a trade name of Rohm and Haas Company) wherein the sodium ion is the exchange cation.
(b) the cationic (weakly acidic) ion exchange resin Amberlite Resin Grade IRP-64 (a trade name of Rohm and Haas Company for a brand of polacrilex resin; herein after generally referred to as IRP-64) wherein the hydrogen ion is the exchange cation. The INN name is polacrilex resin. Polacrilex resin is the methacrylic acid polymer with divinylbenzene.
(c) the cationic (weakly acidic) ion exchange resin Amberlite Resin Grade IRP-88 (a trade name of Rohm and Haas Company) wherein the potassium ion is the exchange cation. The INN name is polacrilin potassium. Polacrilin potassium is the potassium salt of polacrilex resin.

Polacrilin Potassium is a weakly acidic cation exchange resin, and has the ability to bind considerable quantities of water due to its hydrophilic nature.

Taste masked resinate may be prepared by reaction of pharmaceutically acceptable salt of escitalopram with cationic ion exchange resin in a suitable solvent like water, to yield resinate of the pharmaceutically acceptable salt of escitalopram. The reaction may be carried out at about 20-100⁰ C for about 1-10 hrs. The resinate may be used as such or granulated with atleast one pharmaceutical excipient and then used for the preparation of a dosage form.

Typically the taste masked resinate/granules may be prepared as follows;
(a) Disperse or dissolve pharmaceutically acceptable salt of escitalopram in purified water or buffered aqueous solution with the aid of stirring with or without heat.
(b) Disperse 1 to 5 parts of cationic ion exchange resin to the solution/dispersion of (a) with constant stirring. The pH of solution is adjusted with dilute hydrochloric acid to acidic pH (pH 4 to 5 is preferred) to provide favourable condition for the complex between drug and cationic ion exchange resin to be formed. The stirring may be continued for about 1 to 5 hours with or without heating to facilitate complexation between pharmaceutically acceptable salt of escitalopram and ion exchange resin.
(c) Filter the resinate slurry and dry the resinate or adsorb the resinate slurry over microcrystalline cellulose using granulation equipment (e.g. Rapid mixer granulator) and dry the granules in Tray dryer (Results into GRANULES)

The pharmaceutical excipient may be selected from diluents, binders lubricants, disintegrants, flavoring agents, coloring agents, stabilizers, surfactants, glidants, plasticizers, preservatives and sweeteners.

According to another embodiment is provided herewith a taste masked resinate of pharmaceutical acceptable salt of escitalopram which may be used as such or granulated into a resinate granule with atleast one pharmaceutical excipient. The dosage form may be prepared by utilizing the resinate or resinate granules.

The pharmaceutically acceptable salt of escitalopram may be selected form hydrochloride, hydrobromide and oxalate, preferably the pharmaceutically acceptable salt is oxalate.

Taste masked resinate may be prepared by reaction of pharmaceutically acceptable salt of escitalopram with cationic ion exchange resin in a suitable solvent like water, to yield resinate of the pharmaceutically acceptable salt of escitalopram. The reaction may be carried out at about 20-100° C. for about 1-10 hrs.

The Cationic ion exchange resin used in the present invention may be selected from
(a) the cationic (strongly acidic) ion exchange resin Amberlite Resin Grade IRP-69 (a trade name of Rohm and Haas Company) wherein the sodium ion is the exchange cation.
(b) the cationic (weakly acidic) ion exchange resin Amberlite Resin Grade IRP-64 (a trade name of Rohm and Haas Company for a brand of polacrilex resin; herein after generally referred to as IRP-64) wherein the hydrogen ion is the exchange cation. The INN name is polacrilex resin. Polacrilex resin is the methacrylic acid polymer with divinylbenzene.
(c) the cationic (weakly acidic) ion exchange resin Amberlite Resin Grade IRP-88 (a trade name of Rohm and Haas Company) wherein the potassium ion is the exchange cation. The INN name is polacrilin potassium. Polacrilin potassium is the potassium salt of polacrilex resin.

The pharmaceutical excipient may be selected from diluents, binders lubricants, disintegrants, flavoring agents, coloring agents, stabilizers, surfactants, glidants, plasticizers, preservatives and sweeteners.

According to another embodiment of the present invention, a taste masked dosage form of pharmaceutically acceptable salt of escitalopram may comprise coating non-pareil seeds or inert granules with a mixture of pharmaceutically acceptable salt, cationic polymer, an optional polymer and atleast one pharmaceutical excipient.

The non-pareil seeds or inert particles may be selected from water soluble and water insoluble non-fine particles such as directly compressible dibasic calcium phosphate, microcrystalline cellulose, directly compressible sugar such as directly compressible mannitol commercially available as PEARLITOL, starch and the like.

The cationic polymer are polymers with dimethylaminoethyl groups such as Eudragit® E-100 and Eudragit® EPO.

Eudragit E is a cationic polymer based on dimethylaminoethyl methacrylate and neutral methacrylates. It is soluble in gastric fluid as well as in weakly acidic buffer solutions (upto about pH 5).

The optional polymer may be selected from ethylcellulose, cellulose acetate, hydroxypropylmethyl cellulose, hydroxypropyl cellulose, hydroxyethyl cellulose and the like.

The pharmaceutical excipient must be compatible with pharmaceutically acceptable salt of escitalopram. The pharmaceutical excipient is selected from diluents, binders lubricants, disintegrants, flavoring agents, coloring agents, stabilizers, surfactants, glidants, plasticizers, preservatives and sweeteners.

Diluents may be selected from calcium carbonate, calcium phosphate dibasic, calcium phosphate tribasic, calcium sulfate, microcrystalline cellulose, microcrystalline silicified cellulose, powdered cellulose, dextrates, dextrose, fructose, lactitol, lactose anhydrous, lactose monohydrate, lactose dihydrate, lactose trihydrate, mannitol sorbitol, starch, pregelatinized starch, sucrose, talc, xylitol, maltose maltodextrin, maltitol.

Binders may be selected from acacia, alginic acid, carbomer, carboxymethylcellulose calcium, carbomethylcellulose sodium, microcrystalline cellulose, powdered cellulose, ethyl cellulose, gelatin liquid glucose, guar gum, hydroxyethyl cellulos, hydroxypropyl cellulose, hydroxypropylmethyl cellulose, maltodextrin, methylcellulose, ploydextrose, polyethylene oxide, povidone, sodium alginate, starch paste, pregelatinized starch, sucrose, tragacanth, low-substituted hydroxypropyl cellulose, glucose, sorbitol. Suitable fillers are preferably selected from atleast one of starch derivatives, such as corn starch, potato starch or rice starch. Polysaccharides such as dextrins, maltodextrins, dextrates, microcrystalline cellulose, powdered cellulose, mixture of microcrystalline cellulose and guar gum, coprocessed blends of microcrystalline cellulose; and polyhydric alcohols, such as xylitol and sorbitol.

Disintegrants may for example, example, alginic acid, carbon dioxide, carbonxymethylcellulose calcium carboxymethylcellulose sodium, microcrystalline cellulose, powdered cellulose, croscarmelose sodium, crospovidone, sodium docusate, gaur gum, hydroxypropyl cellulose, methylcellulose, polacrilin potassium, poloxamer, povidone, sodium alginate, sodium glycine carbonate, sodium laulyl sulfate, sodium starch glycolate, starch, pregelatinized starch, low-substituted hydroxypropyl cellulose.

Glidants may be, for example, calcium silicate, powdered cellulose, starch, talc, colloidal silicon dioxide.

Lubricants may be selected from magnesium stearate, stearic acid, sodium stearyl fumarate, magnesium lauryl sulphate, talc, polyethylene glycol, and glyceryl behenate.

Suitable sweeteners may be selected from sugars such as sucrose, lactose and glucose; cyclamate and salts thereof; saccharin and salts thereof; and aspartame.

Flavouring agents may be selected from natural or synthetic flavours such as strawberry flavour, wild cherry flavour, green apple flavour, spearmint flavour and peppermint flavour. In one another embodiment of the present invention the process for the preparation of taste masked resinate or taste masked granules or taste masked dosage form is also provided.

A process for the preparation of taste masked dosage form of pharmaceutically acceptable salt of escitalopram comprising
(a) complexing pharmaceutically acceptable salt of escitalopram and cationic ion exchange resin to prepare a resinate of pharmaceutically acceptable salt of escitalopram or adsorbing/coating non-pareil seeds or inert particles with a mixture of pharmaceutically acceptable salt of escitalopram, cationic polymer and optionally other polymer or loading non-pareil seeds or inert particles with pharmaceutically acceptable salt of escitalopram followed by polymer coating with cationic polymer and optionally other polymer(s);
(b) adding atleast one pharmaceutical excipient; and
(c) compressing into tablets.

More particularly, a process for the preparation of taste masked dosage form of escitalopram oxalate comprising
(a) complexing escitalopram oxalate and cationic ion exchange resin to prepare a resinate of escitalopram oxalate or adsorbing/coating non-pareil seeds or inert particles with a mixture of pharmaceutically acceptable salt of escitalopram, cationic polymer and optionally other polymer or loading non-pareil seeds or inert particles with escitalopram oxalate followed by polymer coating with cationic polymer and optionally other polymer(s);
(b) adding atleast one pharmaceutical excipient; and
(c) compressing into tablets.

The dosage form of the present invention may be prepared using conventional techniques employed in the art.

The following examples illustrate preferred embodiments in accordance with the present invention without limiting the scope of the invention.

### EXAMPLES

### Example 1

### Preparation of Resinate of Escitalopram Oxalate

Disperse/dissolve escitalopram oxalate in purified water with the aid of stirring and/or heat to form a solution. Disperse 1 to 4 parts of Polacrillin potassium (ion exchange resin) to the solution/dispersion of escitalopram oxalate with constant stirring. The pH of solution is adjusted with dilute HCl to pH 3-6, stir with or without heating to obtain the resinate. Filter the resinate slurry and keep the resinate for drying.

### Example 2

### Preparation of Orally Disintegrating Tablets of Escitalopram Oxalate of 5, 10 and 20 mg Strengths

| INGREDIENT | MG/TABLET | |
|---|---|---|
| Resinate of Escitalopram | 51.10 mg (equivalent to | |
| oxalate of Example 1 | 10 mg escitalopram base) | |
| Aspartame | 2.25 | mg |
| Flavoring agent | 1.13 | mg |
| Monosodium citrate | 4.50 | mg |
| F-melt type C | 163.77 | mg |
| Magnesium stearate | 2.25 | mg |
| TOTAL | 225 | mg |

1. Cosift resinate/granules along with all other excipients except magnesium stearate through suitable sieve# BSS
2. Mix blend of Step 1 with excipients.
3. Lubricate the blend of Step 2 with magnesium stearate and compress into tablets.

### Example 3

### Preparation of Orally Disintegrating Tablets of Escitalopram Oxalate of 5, 10 and 20 mg Strengths

| INGREDIENT | MG/TABLET | |
|---|---|---|
| Resinate of Escitalopram | 51.10 mg (equivalent to | |
| oxalate of Example 1 | 10 mg escitalopram base) | |
| Aspartame | 2.25 | mg |
| Crospovidone | 4.50 | mg |
| Flavoring agent | 1.13 | mg |
| Monosodium citrate | 4.50 | mg |
| Ludiflash | 159.27 | mg |
| Magnesium stearate | 2.25 | mg |
| TOTAL | 225 | mg |

1. Cosift resinate/granules along with all other excipients except magnesium stearate through suitable sieve# BSS
2. Mix blend of Step 1 with excipients.
3. Lubricate the blend of Step 2 with magnesium stearate and compress into tablets.

### Example 4

### Preparation of Orally Disintegrating Tablets of Escitalopram Oxalate of 5, 10 and 20 mg Strengths

| INGREDIENT | MG/TABLET | |
|---|---|---|
| Resinate of Escitalopram | 51.10 mg (equivalent to | |
| oxalate of Example 1 | 10 mg escitalopram base) | |
| Aspartame | 2.25 | mg |
| Crospovidone | 6.75 | mg |
| Flavoring agent | 1.13 | mg |
| Monosodium citrate | 4.50 | mg |
| Lactose spray dried/mannitol spray dried | 141.27 | mg |
| Microcrystalline cellulose | 15.75 | mg |
| Magnesium stearate | 2.25 | mg |
| TOTAL | 225 | mg |

1. Cosift resinate/granules along with all other excipients except magnesium stearate through suitable sieve# BSS
2. Mix blend of Step 1 with excipients
3. Lubricate the blend of Step 2 with magnesium stearate and compress into tablets.

1. A taste masked resinate comprising a pharmaceutically acceptable salt of escitalopram complexed with a cationic ion exchange resin.

2. A taste masked dosage form comprising a taste masked resinate as claimed in claim 1 and at least one pharmaceutical excipient.

3. The taste masked dosage form as claimed in claim 2, selected from a tablet, a soluble tablet, sprinkle granules or powder for reconstitution in a suspension, a rapidly disintegrating tablet, an orally disintegrating tablet, a rapidly disintegrating film, an orally disintegrating powder for capsules, suspension or sachets, an effervescent tablet, a chewable tablet, a water dispersible tablet, an orodispersible tablet, a chewing gum and a suspension.

4. The taste masked dosage form as claimed in claim 3, wherein the dosage form is an orally disintegrating tablet.

5. The taste masked dosage form as claimed in claim 2, wherein the pharmaceutical excipient is selected from diluents, binders, lubricants, disintegrants, flavoring agents, coloring agents, stabilizers, surfactants, glidants, plasticizers, preservatives and sweeteners.

6. A taste masked dosage form of a pharmaceutically acceptable salt of escitalopram obtained by adsorbing or coating non-pareil seeds or inert particles with a mixture of a pharmaceutically acceptable salt of escitalopram, cationic polymer and optionally other polymer(s).

7. The taste masked dosage form as claimed in claim 6, wherein the cationic polymer is based on dimethylaminoethyl methacrylate and neutral methacrylates.

8. The taste masked dosage form as claimed in claim 6, wherein the other polymer(s) is selected from the group consisting of ethylcellulose, cellulose acetate, hydroxypropylmethyl cellulose, hydroxypropyl cellulose, and hydroxyethyl cellulose.

9. The taste masked dosage form as claimed in claim 6, obtained by further adding at least one pharmaceutical excipient and compressing into tablets.

10. The taste masked dosage form as claimed in claim 9, wherein the dosage form is selected from a tablet, soluble tablet, sprinkle granules or powder for reconstitution in a suspension, a rapidly disintegrating tablet, an orally disintegrating tablet, a rapidly disintegrating film, an orally disintegrating powder for capsules, suspension or sachets, an effervescent tablet, a chewable tablet, a water dispersible tablet, an orodispersible tablet, a chewing gum and a suspension.

11. The taste masked dosage form as claimed in claim 10, wherein the dosage form is an orally disintegrating tablet.

12. A taste masked dosage form of a pharmaceutically acceptable salt of escitalopram obtained by loading a pharmaceutically acceptable salt of escitalopram on non-pareil seeds or inert particles followed by coating with a cationic polymer and optionally other polymer(s).

13. The taste masked dosage form as claimed in claim 12, wherein the catonic polymer is based on dimethylaminoethyl methacrylate and neutral methacrylates.

14. The taste masked dosage form as claimed in claim 12, wherein the other polymer(s) is selected from the group consisting of ethylcellulose, cellulose acetate, hydroxypropylmethyl cellulose, hydroxypropyl cellulose, and hydroxyethyl cellulose.

15. The taste masked dosage form as claimed in claim 12, obtained by further adding at least one pharmaceutical excipient and compressing into tablets.

16. The taste masked dosage form as claimed in claim 15, wherein the pharmaceutical excipient is selected from diluents, binders, lubricants, disintegrants, flavoring agents, coloring agents, stabilizers, surfactants, glidants, plasticizers, preservatives and sweeteners.

17. The taste masked dosage form as claimed in claim 16, wherein the dosage form is selected from a tablet, a soluble tablet, sprinkle granules or powder for reconstitution in a suspension, a rapidly disintegrating tablet, an orally disintegrating tablet, a rapidly disintegrating film, an orally disintegrating powder for capsules, suspension or sachets, an effervescent tablet, a chewable tablet, a water dispersible tablet, an orodispersible tablet, a chewing gum and a suspension.

18. The taste masked dosage form as claimed in claim 17, wherein the dosage form is an orally disintegrating tablet.

19. A taste masked granule of a pharmaceutically acceptable salt of escitalopram comprising the pharmaceutically acceptable salt of escitalopram complexed with a cationic ion exchange resin and at least one pharmaceutical excipient.

20. The taste masked granule as claimed in claim 19, wherein the pharmaceutical excipient is selected from diluents, binders, lubricants, disintegrants, flavoring agents, coloring agents, stabilizers, surfactants, glidants, plasticizers, preservatives and sweeteners.

21. A taste masked granule of a pharmaceutically acceptable salt of escitalopram obtained by adsorbing or coating non-pareil seeds or inert particles with a mixture of a pharmaceutically acceptable salt of escitalopram, cationic polymer and optionally other polymer(s).

22. The taste masked granule as claimed in claim 21, wherein the catonic polymer is based on dimethylaminoethyl methacrylate and neutral methacrylates.

23. The taste masked granule as claimed in claim 21, wherein the other polymer(s) is selected from ethylcellulose, cellulose acetate, hydroxypropylmethyl cellulose, hydroxypropyl cellulose, and hydroxyethyl cellulose.

24. A taste masked orally disintegrating tablet of escitalopram oxalate comprising:
(i) a complex of escitalopram oxalate with a cationic ion exchange resin; and
(ii) at least one pharmaceutical excipient.

25. The taste masked dosage form as claimed in claim 24, wherein the pharmaceutical excipient is selected from diluents, binders, lubricants, disintegrants, flavoring agents, coloring agents, stabilizers, surfactants, glidants, plasticizers, preservatives and sweeteners.

26. A taste masked orally disintegrating tablet of escitalopram oxalate comprising
(i) non-pareil seeds or inert particles coated or adsorbed with a mixture of escitalopram oxalate, cationic polymer and optionally other polymer(s); and
(ii) at least one pharmaceutical excipient.

27. The taste masked dosage form as claimed in claim 26, wherein the catonic polymer is based on dimethylaminoethyl methacrylate and neutral methacrylates.

28. The taste masked dosage form as claimed in claim 26, wherein the other polymer(s) is selected from ethylcellulose, cellulose acetate, hydroxypropylmethyl cellulose, hydroxypropyl cellulose, and hydroxyethyl cellulose.

29. A taste masked dosage form of escitalopram oxlate obtained by loading escitalopram oxalate on non-pareil seeds or inert particles followed by coating with a cationic polymer and optionally other polymer(s).

30. The taste masked dosage form as claimed in claim 29, wherein the catonic polymer is based on dimethylaminoethyl methacrylate and neutral methacrylates.

31. The taste masked dosage form as claimed in claim 29, wherein the other polymer(s) is selected from ethylcellulose, cellulose acetate, hydroxypropylmethyl cellulose, hydroxypropyl cellulose, and hydroxyethyl cellulose.

32. The taste masked dosage form as claimed in claim 29, obtained by further adding at least one pharmaceutical excipient and compressing into tablets.

33. The taste masked dosage form as claimed in claim 32, wherein the pharmaceutical excipient is selected from diluents, binders, lubricants, disintegrants, flavoring agents, coloring agents, stabilizers, surfactants, glidants, plasticizers, preservatives and sweeteners.

34. The taste masked dosage form as claimed in claim 32, wherein the dosage form is selected from a tablet, a soluble tablet, sprinkle granules or powder for reconstitution in a suspension, a rapidly disintegrating tablet, an orally disintegrating tablet, a rapidly disintegrating film, an orally disintegrating powder for capsules, suspension or sachets, an effervescent tablet, a chewable tablet, a water dispersible tablet, an orodispersible tablet, a chewing gum and a suspension.

35. The taste masked dosage form as claimed in claim 34, wherein the dosage form is an orally disintegrating tablet.

36. A process for the preparation of a taste masked dosage form of a pharmaceutically acceptable salt of escitalopram comprising:
(a) complexing a pharmaceutically acceptable salt of escitalopram and a cationic ion exchange resin to prepare a resinate of the pharmaceutically acceptable salt of escitalopram or adsorbing/coating non-pareil seeds or inert particles with a mixture of a pharmaceutically acceptable salt of escitalopram, a cationic polymer and optionally other polymer(s) or loading non-pareil seeds or inert particles with a pharmaceutically acceptable salt of escitalopram followed by polymer coating with a cationic polymer and optionally other polymer(s);
(b) adding at least one pharmaceutical excipient; and
(c) compressing into tablets.

37. The process for the preparation of a taste masked dosage form as claimed in claim 36, wherein the cationic polymer is based on dimethylaminoethyl methacrylate and neutral methacrylates.

38. The process for the preparation of a taste masked dosage form as claimed in claim 36, wherein the pharmaceutical excipient is selected from diluents, binders, lubricants, disintegrants, flavoring agents, coloring agents, stabilizers, surfactants, glidants, plasticizers, preservatives and sweeteners.

39. A process for the preparation of a taste masked dosage form of escitalopram oxalate comprising:
(a) complexing escitalopram oxalate and a cationic ion exchange resin to prepare a resinate of the escitalopram oxalate or adsorbing/coating non-pareil seeds or inert particles with a mixture of a pharmaceutically acceptable salt of escitalopram, a cationic polymer and optionally other polymer(s) or loading non-pareil seeds or inert particles with escitalopram oxalate followed by polymer coating with a cationic polymer and optionally other polymer(s);
(b) adding atleast at least one pharmaceutical excipient; and
(c) compressing into tablets.

40. The process for the preparation of a taste masked dosage form as claimed in claim 39, wherein the cationic polymer is based on dimethylaminoethyl methacrylate and neutral methacrylates.

41. The process for the preparation of a taste masked dosage form as claimed in claim 39, wherein the pharmaceutical excipient is selected from diluents, binders, lubricants, disintegrants, flavoring agents, coloring agents, stabilizers, surfactants, glidants, plasticizers, preservatives and sweeteners.
